(19) United States
(12) Patent Application Publication
(10) Pub. No.: US 2023/0390408 A1
(43) Pub. Date: Dec. 7, 2023

(54) LINKER COMPOUNDS COMPRISING AMIDE BONDS

(71) Applicant: MPEG LA, L.L.C., Chevy Chase, MD (US)

(72) Inventors: Jonathan Miles Brown, Silver Spring, MD (US); Kristin K.H. Neuman, Larchmont, NY (US)

(21) Appl. No.: 18/032,118

(22) PCT Filed: Oct. 14, 2021
(86) PCT No.: PCT/US2021/055085

§ 371 Date: Apr. 14, 2023

Related U.S. Application Data
(60) Provisional application No. 63/093,062, filed on Oct. 16, 2020.

(51) Int. Cl. A61K 47/65; C07K 5/062; C07K 5/072; C07K 5/103; C07K 5/078

(52) U.S. Cl. CPC A61K 47/65; C07K 5/06026; C07K 5/06052; C07K 5/06113; C07K 5/1008; C07K 5/06165

(57) ABSTRACT

Various embodiments provide a homo-bivalent linker compound comprising identical functional groups at either end, methods of making such linker compounds, and methods of using the linker compounds.

### INCORPORATION BY REFERENCE TO ANY PRIORITY APPLICATIONS

Any and all applications for which a foreign or domestic priority claim is identified in the PCT Request as filed with the present application are hereby incorporated by reference.

### FIELD OF THE DISCLOSURE

The present disclosure relates to a compound, method of making the compound, and related uses of the compound as a linking agent for oligonucleotides and other chemical and biological substances.

### BACKGROUND

Preparation of therapeutics in multimeric form can be advantageous because of enhanced bioavailability and uptake.

Bioconjugates (or multi-conjugates) comprise covalent linkages of at least two chemical or biological substances intended for delivery into a cell or tissue. Bioconjugates have a variety of functions, such as in labeling, imaging, and tracking molecular and cellular events, delivering drugs to targeted cells, and as diagnostic or therapeutic agents. Nonlimiting examples of bioconjugates include the coupling of a small molecule (e.g., biotin) to a protein, protein-protein conjugates (e.g., an antibody coupled to an enzyme), antibody drug conjugates (ADCs) (e.g., a monoclonal antibody conjugated to a cytotoxic small molecule), radio-immunoconjugates (e.g., a monoclonal antibody conjugated to a chelating agent), vaccines (e.g., haptens conjugated to carrier proteins), antibodies conjugated to nanoparticles and non-cytotoxic drugs (e.g., peptides), biomolecules conjugated to elements or derivatives thereof (e.g., TGF-β conjugated to iron oxide nanoparticles), and oligonucleotide therapeutic agents conjugated to cell-targeting moieties.

The preparation of therapeutic agents in the form of bioconjugates can produce advantageous effects on the pharmacokinetics and bioavailability of the agents, their intracellular uptake, and ultimately their pharmacodynamics and efficacy. In many instances, it is desirable for some or all of the therapeutic agents within a bioconjugate to be liberated within the target cell, either upon delivery or upon some predetermined time thereafter. This requires the individual agents to be coupled together by linking agents that are cleavable within the cell, oftentimes relying upon innate, intracellular species such as enzymes to perform the cleaving, or upon other innate conditions within the cell.

A variety of cleavable linkers have been employed, including for example short sequences of single-stranded unprotected nucleotides such as dTdTdTdT and dCdA, which are cleaved by intracellular nucleases, and disulfide-based linkers which are cleaved by the reductive environment inside the cell.

However, these types of cleavable linkers can, in certain instances, present challenges in the context of therapeutic multi-conjugates. For example, nuclease cleavable linkers positioned immediately adjacent to a therapeutic oligonucleotide may impact the cleavability of the linker, the activity of the oligo, or both.

Where a disulfide linkage is employed in a cleavable linker compound, the formation of the disulfide bond by reaction of two thiols can lead to mixtures of products, especially with hetero systems. To avoid this problem, an alternative approach is to use an intermediate linking agent capable of reacting with thiol moieties which also contains a preformed internal disulfide bond. Such a linker is dithiobismaleimidoethane (DTME) which has an internal disulfide group and two terminal maleimide groups, each capable of reacting with a thiol group on another molecule.

DTME is normally used as a bivalent linker to link two identical thiolated entities to produce a homo-dimeric derivative. However, it has also been used to generate hetero-dimeric species via a monomeric intermediate wherein only one of the two maleimide moieties is allowed to react with a thiolated molecule. The resulting mono-DTME intermediate is then reacted with a second thiolated moiety to create a DTME linked hetero-dimer. This technique for the synthesis of a hetero-dimer is described in WO 2016/205410.

This methodology has been used to create multimeric oligonucleotides up to octamer in size in both homo-and hetero-multimeric forms.

However, certain aspects of disulfide bonds may be non-optimal for use in the synthesis of chemical compounds in general and of multi-conjugates in particular. For instance, it is not possible to maintain an internal disulfide group in a synthetic intermediate while simultaneously reducing a terminal disulfide to a thiol for subsequent linking reactions. Further, disulfide-linked molecules have been reported to dissociate and/or cross react with other thiolated species. In addition, long-term storage of disulfide-containing molecules can be problematic due to the potential for oxidation and subsequent cleavage of the disulfide bond.

There is therefore a need for additional methods and materials to act as linkers, which retain the advantages of cleavable linkers such as DTME without the perceived drawbacks of disulfide-containing molecules, in the assembly and synthesis of chemical compounds, including for example therapeutic agents and specifically including multi-conjugates of therapeutic agents.

The present disclosure provides provides linkers that are cleavable by intracellular proteases.

Linkers are prepared using chemistries that would otherwise be incompatible with those used to prepare therapeutics such as oligonucleotide agents, for instance using phosphoroamidite.

### SUMMARY OF THE DISCLOSURE

Various embodiments provide a homo-bivalent linker compound comprising identical functional end groups joined by a linking group comprising at least one amide bond, methods of making such linker compounds, and methods of using the linker compounds, as summarized in the claims below.

The disclosure provides for a homo-bivalent linker compound comprising identical functional groups at either end, wherein said functional groups are joined by a linking group comprising at least one amide bond.

In some embodiments, the homo-bivalent linker compound comprises Structure:

(X)-<--->-□-<--->-(X) (Structure 1);

wherein, (X) is a function group; each <---> is independently a spacer group, which may be present or absent; and □ is a linking group comprising at least one amide bond.

In some embodiments, the linking group □ in Structure 1 comprises Structure 2:

R-Aₐ-B_{b}-C_{c}-D_{d}-R′ Structure 2

The disclosure provides for a branched linker compound of Structure 15:

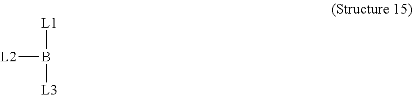

Wherein B is a trivalent moiety; each of L1, L2 and L3 is a branch group; and at least one of LI, L2 and L3 is formed by the joining of B to a homo-bivalent linker compound as disclosed herein; optionally at least two of L1, L2 and L3 are, independently, formed by the joining of B to a homo-bivalent linker compound as disclosed herein; optionally each of L1, L2 and L3 are, independently, formed by the joining of B to a homo-bivalent linker compound as disclosed herein.

The disclosure provides for a multi-conjugate comprising two or more biological moieties joined together by covalent bonds, wherein at least one covalent bond within the multi-conjugate is formed by reaction with a linker compound.

The disclosure provides for a method for synthesizing a multi-conjugate disclosed herein, comprising the steps of reacting a homo-bivalent linker compound as disclosed herein with a first and a second biological moiety, under reaction conditions that promote the formation of a covalent bond between the first biological moiety and the linker compound and a covalent bond between the second biological moiety and the linker compound.

The disclosure provides for a compound comprising a homo-bivalent linker substituted on one end by a biological moiety, wherein the other end of the homo-bivalent linker is unsubstituted, and wherein the compound is at least 75%, 80, 85, 90, 95, 96, 97, 98, 99, or 100% pure

The disclosure provides for a pharmaceutical composition comprising the multi-conjugate as disclosed herein.

The disclosure provides for a method for treating a subject in need of treatment to ameliorate, cure, or prevent the onset of a disease or disorder, the method comprising administering to the subject an effective amount of the multi-conjugate as disclosed herein.

The disclosure provides for a method for modulating gene expression in a cell, in vitro or in vivo, the method comprising delivering to the cell an effective amount of a multi-conjugate as disclosed herein, wherein the multi-conjugate comprises at least one biological moiety that has the effect of modulating gene expression.

The disclosure provides for a method for delivering, in vitro or in vivo, two or more biological moieties to a cell per internalization event, comprising administering to the cell a multi-conjugate as disclosed herein.

The disclosure provides for a method of treating a disease or condition in a subject comprising the step of administering to the subject an effective amount of a pharmaceutical composition comprising an active pharmaceutical ingredient joined by a covalent bond formed by reaction with a linker compound as disclosed herein.

The disclosure provides for a homo-bivalent linker compound comprising:

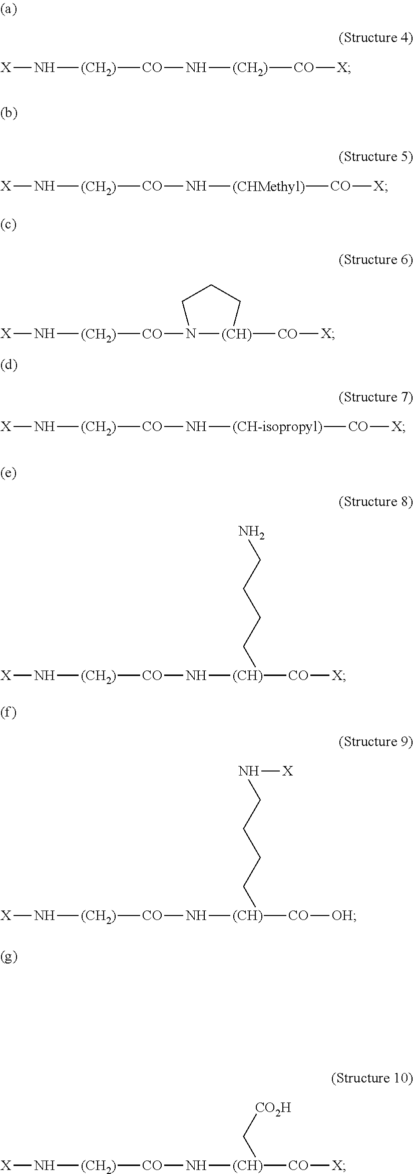

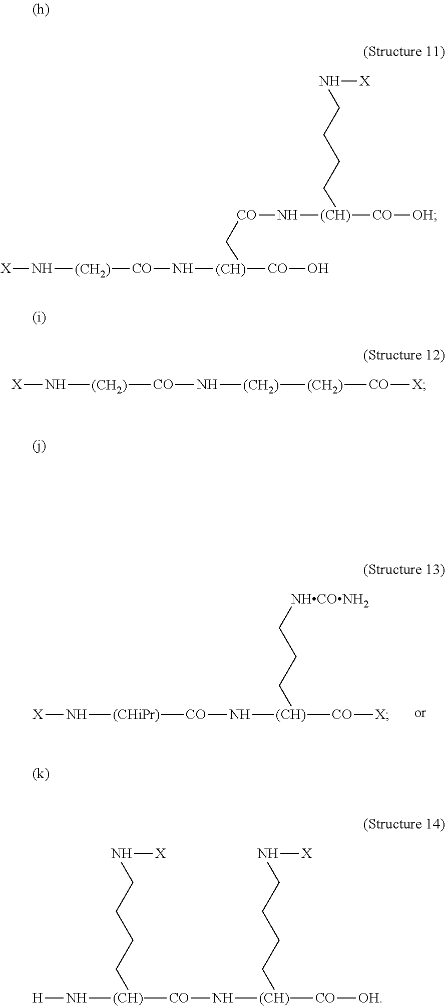

These and other embodiments are described in greater detail below.

While the disclosure comprises embodiments in many different forms, there will herein be described in detail several specific embodiments with the understanding that the present disclosure is to be considered as an exemplification of the principles of the technology and is not intended to limit the disclosure to the embodiments illustrated.

### DETAILED DESCRIPTION

The disclosures of any patents, patent applications, and publications referred to herein are hereby incorporated by reference in their entireties into this application in order to more fully describe the state of the art known to those skilled herein as of the date of the disclosure described and claimed herein.

### Amides

The disclosure provides for a homo-bivalent linker compound comprising identical functional end groups joined by a linking group comprising at least one amide bond.

As used herein, the term “amide” has its ordinary meaning as understood by those skilled in the art. It refers to a compound with the general formula RC(═O)NR′R″, wherein R, R′ and R″ are organic groups or hydrogen bonds.

An amide group is referred to as a “peptide bond” or a “eupeptide bond” when it is formed by the coupling of two amino acids through the backbone (non-side chain) carboxyl group of one amino acid and the backbone (non-side chain) amino group of another amino acid.

An “isopeptide bond” is another type of amide bond, formed by the coupling of a carboxyl group on one amino acid and an amino group on another amino acid, wherein at least one of these coupling groups is part of the side chain of one of the amino acids.

### Amino Acids

As used herein, the term “amino acid” has its ordinary meaning as understood by those skilled in the art. It refers to an organic compound that contains amine and carboxyl functional groups, and a side chain specific to each amino acid. As provided herein, amino acids can be naturally occurring or non-naturally occurring (synthetic), or derivatives thereof. One example of naturally occurring amino acids are the group known as the proteinogenic amino acids, which are used in the synthesis of naturally occurring polypeptides and proteins.

The disclosure provides, in some aspects, for amino acids designated as alpha, beta, gamma, and delta amino acids based on the attachment location of the core amine group, namely the alpha carbon, the beta carbon, the gamma carbon or the delta carbon next to the core carboxyl group. For example, the genetic formula for an alpha amino acid is H₂NCHRCOOH, wherein R is a side chain.

### Homo-Bivalent Linker Compounds Comprising Amide Bonds

The disclosure provides for a homo-bivalent linker compound comprising identical functional end groups joined by a linking group comprising at least one amide bond.

As used herein, the term “homo-bivalent linker compound” has its ordinary meaning as understood by those skilled in the art. It is a molecule of medium molecular weight (e.g., 100-1500 daltons), usually linear in structure, bearing two identical functional groups.

In some aspects of the disclosure, the functional end groups are maleimide, azide, alkyne, activated carboxyl or amine. Other functional end groups suitable for use in connection with this disclosure will be known to those skilled in the art.

In various aspects of the disclosure, the coupling of a functional end group to the linking group of the homo-bivalent linker compound is mediated by aspacer group. As used herein, the term “spacer group” has its ordinary meaning as understood by those skilled in the art.

In some aspects of the disclosure, a spacer group is alkyl, alkoxy, cyclyl, heterocyclyl, aryl, heteroaryl, or substituted versions thereof. In other aspects, the spacer group is C₁₋₁₀ alkyl, C₁₋₁₀ alkoxy, 5-10 membered aryl, 5-10 membered heteroaryl, 5-10 membered heterocyclyl, (C₁₋₁₀ alkyl)-(5-10 membered aryl), (C₁₋₁₀ alkyl)-(5-10 membered heteroaryl), or (C₁₋₁₀ alkyl)-(5-10 membered heterocyclyl). In still further aspects, the spacer group is C₂ to C₆ alkyl, ethylene glycol, triethylene glycol, or 1,4-phenylene. Other suitable spacer groups will be known to those of skill in the art.

In some aspects of the disclosure, the at least one amide bond in the linking group is a eupeptide bond, in other aspects it is an isopeptide bond, and in aspects of the disclosure in which the homo-bivalent linker compound comprises two or more amide bonds, the bonds may be eupeptide, isopeptide, or any combination of the two.

In an embodiment of the homo-bivalent linker compound at least one amide bond is formed from the joining of two amino acids, each of which may be naturally occurring or non-naturally occurring; an alpha, beta, gamma, or delta amino acid; or a proteogenic amino acid; and in the case of a linker compound comprising two or more amide bonds formed from amino acids, the amino acids may be any combination of the foregoing.

In various embodiments of the homo-bivalent linker compound, the compound comprises at least one Alanine, Proline, Valine, Lysine, Aspartic Acid, Citrulline, or Beta-Alanine.

In some aspects of the disclosure, the homo-bivalent linker compound comprises Structure 1:

(X)-<--->-□-<--->-(X) (Structure 1);

wherein,
(X) is a functional group;
each <---> is independently a spacer group, which may be present or absent,
and
□ is a linking group comprising at least one amide bond.

In some embodiments of a homo-bivalent linker compound according to Structure 1, only one spacer group is present in the compound. These embodiments are represented as Structure 1a and Structure 1b as follows:

(X)-(---)-□-(X) (Structure 1a)

(X)-□-(---)-(X) (Structure 1b);

wherein in each of Structures 1a and 1b; (X) is a functional group; (---) is a spacer group; and □ is a linking group comprising at least one amide bond.

In some embodiments of a homo-bivalent linker compound according to Structure 1, each of the spacer groups is present in the compound. These embodiments are represented as Structure 1c as follows:

(X)-(---)-□-(--)-(X) (Structure 1c);

wherein (X) is a functional group; each of (---) is independently a spacer group; and □ is a linking group comprising at least one amide bond.

In some embodiments of a homo-bivalent linker compound according to Structure 1, neither of the spacer groups is present in the compound. These embodiments are represented as Structure 1d as follows:

(X)-□-(X) (Structure 1d);

wherein (X) is a functional group; and □ is a linking group comprising at least one amide bond.

In various embodiments of the homo-bivalent linker compound of Structures 1, 1a, 1b, 1c and 1d, the functional group X is maleimide, azide, alkyne, activated carboxyl or amine. Other functional groups suitable for use in connection with these embodiments will be known to those skilled in the art.

In various embodiments of the homo-bivalent linker compound of Structures 1, 1a, 1b and 1c, each of the spacer groups present in the compound is, independently, alkyl, alkoxy, cyclyl, heterocyclyl, aryl, heteroaryl, or substituted versions thereof. In other embodiments, each of the spacer groups present in the compound is, independently, C₁₋₁₀ alkyl, C₁₋₁₀ alkoxy, 5-10 membered aryl, 5-10 membered heteroaryl, 5-10 membered heterocyclyl, (C₁₋₁₀ alkyl)-(5-10 membered aryl), (C₁₋₁₀ alkyl)-(5-10 membered heteroaryl), or (C₁₋₁₀ alkyl)-(5-10 membered heterocyclyl). In still further embodiments, each of the spacer groups present in the compound is, independently, C₂ to C₆ alkyl, ethylene glycol, triethylene glycol, or 1,4-phenylene. Other suitable spacer groups will be known to those of skill in the art.

In various embodiments of the homo-bivalent linker compound of Structures 1, 1a, 1b, 1c and 1d, □ is a linking group comprising one, two, three, or more than 3 amide bonds. In some embodiments, each amide bond is, independently, a eupeptide bond or an isopeptide bond.

In various embodiments of the homo-bivalent linker compound of Structures 1, 1a, 1b, 1c and 1d, □ is a linking group comprising at least one amide bond formed from the linkage of two amino acids; two amide bonds formed from the linkage of three amino acids; three amide bonds formed from the linkage of four amino acids; etc. In some embodiments, each of the amino acids is, independently, Glycine, Alanine, Proline, Valine, Lysine, Aspartic Acid, Citrulline, or Beta-Alanine.

In some aspects of the disclosure, the homo-bivalent linker compound of Structures 1, 1a, 1b, 1c and 1d, the linking group comprising at least one amide bond (□) comprises Structure 2:

R-Aa-Bb-Cc-Dd-R′ (Structure 2)
wherein:
R is H, or is absent;
R′ is OH, or is absent;
each of a, b, c, and d is independently 0 or 1, with the proviso that the sum of a+b+c+d is greater than or equal to 2; and
each of A, B, C and D independently comprises Structure 3:

N▴ [symbol] -(CH [symbol] )w-(CH [symbol] )x-(CH [symbol] )y-(CH [symbol] )z-CO-▾(Structure 3)
wherein:
   each of w, x, y, and z are independently 0 or 1, with the proviso that the sum of w+x+y+z is greater than or equal to 1;
   each ▴ is independently H, H₂, alkyl, alkoxy, alkyl carboxy, alkyl carboxamide, alkyl amino, alkyl sulfate, aryl, aryl carboxy, aryl carboxamide, aryl amino, aryl sulfate, or is absent;
   each of [symbol] , [symbol] , [symbol] , [symbol] , and [symbol] is independently present or absent, and if present designates a terminus of a cyclic group as follows:
   [symbol] designates the N in N [symbol] as a terminus;
   [symbol] designates the C in ( [symbol] )w as a terminus;
   [symbol] designates the C in ( [symbol] )x as a terminus;
   [symbol] designates the C in ( [symbol] )y as a terminus; and
   [symbol] designates the C in ( [symbol] )z as a terminus;
   with the proviso that each Structure 3 independently contains zero, one or two cyclic groups, the termini of each cyclic group being selected from:
   [symbol] as a first terminus and [symbol] , [symbol] , [symbol] , or [symbol] as a second terminus;
   [symbol] as a first terminus and [symbol] , [symbol] , or [symbol] as a second terminus;
   [symbol] as a first terminus and [symbol] or [symbol] as a second terminus; or
   [symbol] as a first terminus and [symbol] as a second terminus;
   with the further proviso that:
   if [symbol] is present, then [symbol] is absent;
   if [symbol] is present, then [symbol] is absent;
   if [symbol] is present, then [symbol] is absent;
   if [symbol] is present, then [symbol] is absent;
   each cyclic group that is present in Structure 3 further comprises, in addition to its respective termini, a middle section between the termini, Y; and each Y is independently alkyl, alkoxy, alkyl carboxy, alkyl carboxamide, alkyl amino, or alkyl sulfate;
   each of [symbol] , [symbol] , [symbol] , and [symbol] are independently present or absent, and if present are H, OH, alkyl, alkyl carboxy, alkyl carboxamide, alkyl amino, alkoxy, thioalkyl, alkylthioalkyl, aryl, or heteroaryl;
   each of [symbol] , [symbol] , [symbol] , and [symbol] are, where present, optionally bonded to a functional end group X, with or without a spacer group (---)
   each ▾ is independently OH, alkyl, alkoxy, alkyl carboxy, alkyl carboxamide, alkyl amino, alkyl sulfate, aryl, aryl carboxy, aryl carboxamide, aryl amino, aryl sulfate, or is absent; and
with the proviso that the resulting homo-bivalent linker compound contains a total of only two functional end groups X, in keeping with the compound being a homo-bivalent linker compound.

In various embodiments of the homo-bivalent linker compound, the linking group comprising at least one amide bond (□) comprises at least two amino acids. In some embodiments, each of the amino acids is naturally occurring or non-naturally occurring. In some embodiments, each of the amino acids is an alpha, beta, gamma, or delta amino acid. In some embodiments, at least one of the amino acids is a proteogenic amino acid; or each of the amino acids is a proteogenic amino acid.

In some embodiments of the homo-bivalent linker compound, the linking group comprising at least one amide bond (□) comprises a eupeptide bond formed by the joining of Glycine to Glycine according to Structure 4; Glycine to Alanine according to Structure 5; Glycine to Proline according to Structure 6; Glycine to Valine according to Structure 7; Glycine to Lysine according to Structure 8; Glycine to Lysine according to Structure 9; Glycine to Aspartic Acid according to Structure 10; Glycine to Beta-Alanine according to Structure 12; Valine to Citrulline according to Structure 13; Lysine to Lysine according to Structure 14.

In some embodiments of the homo-bivalent linker compound, the linking group comprising at least one amide bond (□) comprises a eupeptide bond and an isopeptide bond formed by the joining of Glycine, Aspartic Acid, and Lysine, according to Structure 11.

The disclosure provides for homo-bivalent linker compounds that are at least 75, 80, 85, 90, 95, 96, 97, 98, 99, or 100% pure. In some embodiments, the linker compound is about 85-95% pure. In some embodiments, the linker compound is greater than or equal to 75% pure; greater than or equal to 85% pure; or greater than or equal to 95% pure.

### Branched Linker Compounds Comprising Amide Bonds

The disclosure provides for a branched linker compound of Structure 15;

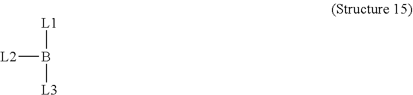

wherein:
B is a trivalent moiety;
each of L1, L2 and L3 is a branch group; and
at least one of L1, L2 and L3 is formed by the joining of B to a homo-bivalent linker compound as defined in any of Structures 1 to 14.

The trivalent moiety (B) within the branched linker compound is derived from a starting material having three functional end groups available for reaction, examples of which include substituted ammonias (HNR¹R²R³) such as tris(hydroxyalkyl)ammonium; certain triols and their derivatives such as tris(hydroxymethyl)aminomethane, glycerol, 1-thioglycerol, 1,3-(2-hydroxymethyl)-propanediol, trihydroxybenzene and deoxyribose.

In some embodiments of the branched linker compound of Structure 15, at least two of L1, L2 and L3 are, independently, formed by the joining of B to a homo-bivalent linker compound as defined in any of Structures 1 to 14.

In some embodiments of the branched linker compound of Structure 15, each of L1, L2 and L3 are, independently, formed by the joining of B to a homo-bivalent linker compound as defined in any of Structures 1 to 14.

The disclosure provides branched linker compounds that are at least 75, 80, 85, 90, 95, 96, 97, 98, 99, or 100% pure. In some embodiments, the linker compound is about 85-95% pure. In some embodiments, the linker compound is greater than or equal to 75% pure; greater than or equal to 85% pure; or greater than or equal to 95% pure.

### Conjugates and Multi-Conjugates Comprising the Linker Compounds

The disclosure provides for a multi-conjugate comprised of two or more biological moieties joined together by covalent bonds, wherein at least one covalent bond within the multi-conjugate is formed by reaction with a linker compound of any of Structures 1 to 15, or as recited in any of claims 1 to 53, which follow.

In some embodiments of the multi-conjugate, each of the biological moieties is joined to another biological moiety by a linker compound of any of Structures 1 to 15, or as recited in any of claims 1 to 53.

As used herein, the term “biological moiety” has its ordinary meaning as understood by those skilled in the art. It refers to chemical entities that are biologically active or inert when delivered into a cell or organism.

In many instances, a biological moiety will produce a biological effect or activity within the cell or organism to which it is delivered; and oftentimes the biological effect or activity is detectable or measurable. In other instances, a biological moiety may be selected to augment or enhance the biological effect or activity of another biological moiety with which it is delivered. In still other instances, a biological moiety may be selected for use in a method for synthesizing a synthetic intermediate or multi-conjugate.

Examples of biological moieties include but are not limited to nucleic acids, amino acids, peptides, proteins, lipids, carbohydrates, carboxylic acids, vitamins, steroids, lignins, small molecules, organometallic compounds, or derivatives of any of the foregoing.

In some aspects of the disclosure, the multi-conjugate comprises two, three, four, five, or six biological moieties.

In some embodiments of the multi-conjugate, each biological moiety is, independently, a nucleic acid, peptide, protein, lipid, carbohydrate, carboxylic acid, vitamin, steroid, lignin, small molecule, organometallic compound, or a derivative of any of the foregoing.

In some embodiments of the multi-conjugate, at least two biological moieties are oligonucleotides; optionally the at least two oligonucleotides are adjacent one another in the multi-conjugate; and optionally each of the oligonucleotides is 15-30, 17-27, 19-26, or 20-25 nucleotides in length.

In some embodiments of the multi-conjugate, at least one of the biological moieties is a double-stranded RNA; optionally an siRNA, a saRNA, or a miRNA.

In some embodiments of the multi-conjugate at least one of the biological moieties is a single-stranded RNA, optionally an antisense oligonucleotide.

In some embodiments of the multi-conjugate, each of the biological moieties is a double-stranded siRNA.

In some embodiments of the multi-conjugate, at least one biological moiety is a protein, a peptide, or a derivative thereof.

Some embodiments of the multi-conjugate will have one or more covalent bonds formed by reaction with a homo-bivalent linker compound having maleimide functional groups, each of which, upon reaction, is independently

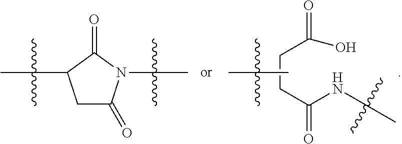

The homo-bivalent linker compound, as described above in all of its various embodiments, may be used in a linking or conjugation reaction to join various chemical or biological compounds.

Conjugates of chemical or biological compounds include, but are not limited to, antibody drug conjugates comprising an antibody or antibody fragment conjugated to a drug agent, including but not limited to a small molecule drug or an oligonucleotide therapeutic; other protein conjugates; and oligonucleotide conjugates. In an embodiment, the conjugates comprise oligonucleotides, polypeptides, or proteins involved in gene editing systems such as CRISPR/Cas, TALES, TALENS, and zinc finger nucleases (ZFNs).

In other embodiments, the linker compound may be used in a series of linker or conjugation reactions to join multiple chemical or biological agents to form a multi-conjugate.

In an embodiment, the multiconjugate is a multimeric oligonucleotide comprised of two or more oligonucleotide “subunits” (each individually a “subunit”) linked together via covalent bonds formed by reaction with at least one linker compound as described herein, wherein the subunits may be multiple copies of the same subunit or differing subunits.

The conjugates, multiconjugates, and multimeric oligonucleotides may comprise all known types of nucleic acids, double-stranded and single-stranded, including for example, siRNAs, saRNAs, miRNAs, antagomirs, CRISPR RNAs, long noncoding RNAs, piwi-interacting RNA, messenger RNA, short hairpin RNA, aptamers, ribozymes, and antisense oligonucleotides (for example, gapmers)

The present disclosure relates to a multimeric oligonucleotide comprising subunits, wherein each of the subunits is independently a single-stranded or double-stranded oligonucleotide, and one or more of the subunits is joined to another subunit by covalent bonds formed by reaction with a linker compound as described herein, including but not limited to a linker compound represented by any of Structures 1-15.

In any of the foregoing multimeric oligonucleotides, at least two subunits are substantially different; alternatively, all of the subunits in the multimeric oligonucleotide are substantially different from one another.

In any of the foregoing multimeric oligonucleotides, at least two subunits are the same; alternatively, all of the subunits in the multimeric oligonucleotide are the same.

In any of the foregoing embodiments, the multimeric oligonucleotide comprises two, three, four, five or six subunits.

In any of the foregoing multimeric oligonucleotides, each subunit is 15-30, 17-27, 19-26, or 20-25 nucleotides in length.

In any of the foregoing multimeric oligonucleotides, one or more of the subunits are a double-stranded RNA or DNA; alternatively all of the subunits are a double-stranded RNA or DNA; alternatively one, or more, or all of the subunits are siRNA, saRNA, or miRNA.

In any of the foregoing multimeric oligonucleotides, one or more of the subunits are an RNA or a DNA comprising a self-hybridizing, double-stranded segment, e.g., but not limited to an aptamer.

In any of the foregoing multimeric oligonucleotides, one or more of the subunits are a single-stranded RNA or DNA; alternatively all of the subunits are a single-stranded RNA or DNA.

In any of the foregoing multimeric oligonucleotides, the subunits comprise a combination of single-stranded and double-stranded oligonucleotides.

### Methods for Synthesizing a Multi-Conjugate

The disclosure provides methods for synthesizing a multi-conjugate comprising the steps of reacting a homo-bivalent linker compound with a first and a second biological moiety, under reaction conditions that promote the formation of a covalent bond between the first biological moiety and the linker compound and a covalent bond between the second biological moiety and the linker compound.

In an embodiment of the method, the first biological moiety and the second biological moiety are the same and the coupling of each of the biological moieties to the homo-bivalent linker compound is performed simultaneously.

In an embodiment of the method wherein the first biological moiety and the second biological moiety are different, the coupling of each of the biological moieties to the homo-bivalent linker compound is performed sequentially under reaction conditions that substantially favor the formation of an isolatable intermediate comprising the homo-bivalent linker monosubstituted with the first biological moiety and substantially prevent dimerization of the first biological moiety.

In an embodiment of the sequential method, the coupling of the homo-bivalent linker compound to the first biological moiety is carried out in a dilute solution of the first biological moiety with a stoichiometric excess of the homo-bivalent linker compound.

In an embodiment of the sequential method, the coupling of the homo-bivalent linker compound to the first biological moiety is carried out with a molar excess of the homo-bivalent linker compound of at least about 5, 10, 15, 20, 25, 30, 35, 40, 45, 50, or 100.

In an embodiment of the sequential method, the coupling of the homo-bivalent linker compound to the first biological moiety is carried out with a molar excess of the homo-bivalent linker compound of about 5, 10, 15, 20, 25, 30, 35, 40, 45, 50, or 100.

In an embodiment of the sequential method, the coupling of the homo-bivalent linker compound to the first biological moiety is carried out in a solution comprising water and a water miscible organic co-solvent. In a further embodiment, the water miscible organic co-solvent comprises DMF, NMP, DMSO, alcohol, or acetonitrile. In a further embodiment, the water miscible organic co-solvent comprises about 10, 15, 20, 25, 30, 40, or 50% (v/v) of the solution. In a still further embodiment, the coupling of the homo-bivalent linker compound to the first biological moiety is carried out at a pH of below about 7, 6, 5, or 4. In another embodiment, the coupling of the homo-bivalent linker compound to the first biological moiety is carried out at a pH of about 7, 6, 5, or 4.

In an embodiment of the sequential method, the coupling of the homo-bivalent linker compound to the first biological moiety is carried out in a solution comprising an anhydrous organic solvent. In a further embodiment, the anhydrous organic solvent comprises dichloromethane, DMF, DMSO, THF, dioxane, pyridine, alcohol, or acetonitrile.

In an embodiment of any of the methods for synthesizing a multi-conjugate, the yield of the multi-conjugate is at least 75, 80, 85, 90, 95, 96, 97, 98, 99, or 100%.

In an embodiment of any of the methods for synthesizing a multi-conjugate, the purity of the compound is at least 75, at least 75, 80, 85, 90, 95, 96, 97, 98, 99, or 100%.

The sequential method for synthesizing a multi-conjugate produces, as a synthetic intermediate, a compound comprising a homo-bivalent linker compound that is substituted on one end by a biological moiety and the other end of the homo-bivalent linker compound is unsubstituted (a mono-substituted homo-bivalent linker). The mono-substituted homo-bivalent linker so produced is at least 75%, 80, 85, 90, 95, 96, 97, 98, 99, or 100% pure.

In various embodiments of the mono-substituted homo-bivalent linker, the biological moiety is a nucleic acid, peptide, protein, lipid, carbohydrate, carboxylic acid, vitamin, steroid, lignin, small molecule, organometallic compound, or a derivative of any of the foregoing.

### Pharmaceutical Compositions and Medicaments

The disclosure provides for a pharmaceutical composition comprising a multi-conjugate formed in a synthesis process that utilizes at least one linker compound as described herein, including but not limited to any of Structures 1 to 15, or as recited in any of claims 1 to 50, which follow; and/or comprising a multi-conjugate as recited in any of claims 54 to 63, which follow.

The disclosure further provides a multi-conjugate for use in the manufacture of a medicament, wherein the multi-conjugate is formed in a synthesis process that utilizes at least one linker compound as described herein, including but not limited to of any of Structures 1 to 15, or as recited in any of claims 1 to 50, which follow; and/or a multi-conjugate as recited in any of claims 54 to 63, which follow.

The present disclosure relates to pharmaceutical compositions comprising an active pharmaceutical ingredient. In an embodiment, the active pharmaceutical ingredient can be joined to another chemical or biological substance by a covalent bond formed by reaction with a linker compound of any of Structures 1-14 and branched, multivalent linkers as described herein including but not limited to Structure 15. The active pharmaceutical ingredient may be a protein, peptide, amino acid, nucleic acid, targeting ligand, carbohydrate, polysaccharide, lipid, organic compound, or inorganic compound.

As used herein, pharmaceutical compositions include compositions of matter, other than foods, that contain one or more active pharmaceutical ingredients that can be used to prevent, diagnose, alleviate, treat, or cure a disease. Similarly, the various compounds or compositions according to the disclosure should be understood as including embodiments for use as a medicament and/or for use in the manufacture of a medicament.

A pharmaceutical composition can include a composition comprising an active pharmaceutical ingredient joined by a covalent bond formed by reaction with a linker compound as described herein, including but not limited to a linker compound of any of Structures 1-15, and a pharmaceutically acceptable excipient. As used herein, an excipient can be a natural or synthetic substance formulated alongside the active ingredient. Excipients can be included for the purpose of long-term stabilization, increasing volume (e.g., bulking agents, fillers, or diluents), or to confer a therapeutic enhancement on the active ingredient in the final dosage form, such as facilitating drug absorption, reducing viscosity, or enhancing solubility. Excipients can also be useful manufacturing and distribution, for example, to aid in the handling of the active ingredient and/or to aid in vitro stability (e.g., by preventing denaturation or aggregation). As will be understood by those skilled in the art, appropriate excipient selection can depend upon various factors, including the route of administration, dosage form, and active ingredient(s).

The pharmaceutical composition can be delivered locally or systemically, and the administrative route for pharmaceutical compositions of the disclosure can vary according to application. Administration is not necessarily limited to any particular delivery system and may include, without limitation, parenteral (including subcutaneous, intravenous, intramedullary, intraarticular, intramuscular, intraperitoneal, intraparenchymal, intracerebroventricular, and intrathecal, cisternal and lombar), rectal, topical, transdermal, or oral. Administration to an individual may occur in a single dose or in repeat administrations, and in any of a variety of physiologically acceptable salt forms, and/or with an acceptable pharmaceutical carrier and/or additive or adjuvant as part of a pharmaceutical composition. Physiologically acceptable formulations and standard pharmaceutical formulation techniques, dosages, and excipients are well known to persons skilled in the art (see, e.g., Physicians' Desk Reference (PDR®) 2005, 59th ed., Medical Economics Company, 2004; and Remington: The Science and Practice of Pharmacy, eds. Gennado et al. 21th ed., Lippincott, Williams & Wilkins, 2005).

Pharmaceutical compositions can include an effective amount of the linker compound or composition (e.g., conjugates and multimeric oligonucleotides comprising the linker compound) according to the disclosure. As used herein, effective amount can be a concentration or amount that results in achieving a particular purpose, or an amount adequate to cause a change, for example in comparison to a placebo. Where the effective amount is a therapeutically effective amount, it can be an amount adequate for therapeutic use, for example an amount sufficient to prevent, diagnose, alleviate, treat, or cure a disease or condition. An effective amount can be determined by methods known in the art. An effective amount can be determined empirically, for example by human clinical trials. Effective amounts can also be extrapolated from one animal (e.g., mouse, rat, monkey, pig, dog) for use in another animal (e.g., human), using conversion factors known in the art. See, e.g., Freireich et al., Cancer Chemother Reports 50 (4):219-244 (1966).

### Methods of using Products Comprising the Linker Compounds

The present disclosure also relates to methods of using compounds containing the above-described linkers in various applications, including but not limited to delivery to cells in vitro or in vivo for the purpose of modulating gene expression, biological research, treating or preventing medical conditions, and/or to produce new or altered phenotypes.

In an embodiment, the disclosure provides a method of treating a disease or condition in a subject by administering to the subject an effective amount of a pharmaceutical composition comprising an active pharmaceutical ingredient joined by a covalent bond formed by reaction with a linker compound as described herein including but not limited to linker compounds according to any of Structures 1-16. In an embodiment, the linker compound in the pharmaceutical composition is or comprises an active pharmaceutical ingredient (e.g., an ASO).

In one aspect, the disclosure provides a method for modulating gene expression, for example to silence, activate or inhibit gene expression, comprising administering an effective amount of a pharmaceutical composition comprising a linker compound, or an active pharmaceutical ingredient joined by a covalent bond formed by reaction with a linker compound, according to any of the linker compounds described herein, including but not limited to Structures 1-16, to a subject in need thereof. In such therapeutic embodiments, the linker compound may be present within or conjugated to an oligonucleotide that modulates gene expression, for example an siRNA, saRNA, miRNA, antagomir, CRISPR RNA, long noncoding RNA, piwi-interacting RNA, messenger RNA, short hairpin RNA, aptamer, ribozyme, or antisense oligonucleotide (for example, a gapmer). In another embodiment, the linker compound may be conjugated to a protein or protein fragment involved in modulating gene expression, for example any of the CRISPR-Cas protein effectors (e.g., Cas9), TALES, TALENS, zinc finger nucleases, or derivatives of any of the foregoing.

As used herein, a “subject” includes, but is not limited to, mammals, such as primates, rodents, and agricultural animals. Examples of a primate subject includes, but is not limited to, a human, a chimpanzee, and a rhesus monkey. Examples of a rodent subject includes, but is not limited to, a mouse and a rat. Examples of an agricultural animal subject includes, but is not limited to, a cow, a sheep, a lamb, a chicken, and a pig

### Methods for Treating Subjects

The disclosure provides a method for treating a subject in need of treatment to ameliorate, cure, or prevent the onset of a disease or disorder, the method comprising administering to the subject an effective amount of the multi-conjugate formed in a synthesis process that utilizes at least one linker compound as described herein, including but not limited to any of Structures 1 to 15, or as recited in any of claims 1 to 50, which follow; and/or comprising a multi-conjugate as described herein, including but not limited to a multi-conjugate recited in any of claims 54 to 63, which follow.

The disclosure provides a method of treating a disease or condition in a subject comprising the step of administering to the subject an effective amount of a pharmaceutical composition comprising an active pharmaceutical ingredient joined by a covalent bond formed by reaction with a at least one linker compound as described herein, including but not limited to any of Structures 1 to 15, or as recited in any of claims 1 to 50, which follow.

### Methods for Modulating Gene Expression

The disclosure provides a method for modulating gene expression in a cell, in vitro or in vivo, the method comprising delivering to the cell an effective amount of a multi-conjugate as described herein, including but not limited to a multi-conjugate as recited in any of claims 54 to 63, which follow, and a multi-conjugate formed in a synthesis process that utilizes at least one linker compound as described herein, including but not limited to any of Structures 1 to 15, or as recited in any of claims 1 to 50, which follow; wherein the multi-conjugate comprises at least one biological moiety that has the effect of modulating gene expression.

In an embodiment of this method, at least one biological moiety in the multi-conjugate silences or reduces gene expression. In an embodiment, the foregoing biological is siRNA, miRNA, or an antisense oligonucleotide.

In an embodiment of this method, at least one biological moiety in the multi-conjugate activates or increases gene expression. In an embodiment, the foregoing biological moiety is saRNA.

### Methods for Delivery to a Cell

The disclosure provides a method for delivering, in vitro or in vivo, two or more biological moieties to a cell per internalization event, comprising administering to the cell a multi-conjugate as described herein, including but not limited to a multi-conjugate as recited in any of claims 54 to 63, which follow, and/or a multi-conjugate formed in a synthesis process that utilizes at least one linker compound of any of Structures 1 to 15, or as recited in any of claims 1 to 50, which follow.

In an embodiment of the method, the multi-conjugate is formulated in a lipid nanoparticle.

In an embodiment of the method, the multi-conjugate is packaged in a viral vector.

In an embodiment of the method, the multi-conjugate comprises a cell- or tissue-targeting ligand.

In an embodiment of the method, the multi-conjugate comprises 3 or more biological moieties in a predetermined stoichiometric ratio.

### Tunable Linker Compounds

The present disclosure relates to linker compounds configured or selected to exhibit higher or lower stability to cleavage by proteases. These enzymes are ubiquitous in the human body and form key parts of metabolic pathways. However, differing proteases with differing activity profiles are present in various cell and tissue types. A key aspect of the disclosed linker compound is lability to certain proteases and simultaneous resistance to others.

The linker compounds described herein are resistant to exoproteases (or exopeptidases) as the linking functional groups at the termini are non-amino acid in nature and hence the whole linker is not susceptible to this class of enzymes. By contrast, the internal linking group comprising at least one amide bond can contain one or more amino acid residues which are susceptible to endo-proteases. This susceptibility can be increased or decreased according to preference by altering the number, type, and position of the amino acid derivatives in the linker compound. Thus, by taking advantage of the higher lability of simple amino acids to endo-proteases, the linker may contain, e.g., a Gly-Gly sequence for rapid cleavage. Alternatively, the internal linker sequence may contain, e.g., synthetic non-proteogenic amino acids for greater stability to endoproteases. In general a higher proportion of synthetic rather than proteogenic amino acids, together with a greater proportion of spacer groups, results in a greater stability of the linker and a corresponding slower rate of cleavage by endo-proteases. And vice versa

In this way the biological characteristics of the linker compound can be “tuned” to the user's requirements.

### Targeting Agents

Drug delivery systems have been designed using targeting ligands or conjugate systems to facilitate delivery to specific cells or tissues. For example, oligonucleotides can be conjugated to cholesterols, sugars, peptides, and other nucleic acids to facilitate delivery into hepatocytes and/or other cell types. Oftentimes, such conjugate systems facilitate delivery into specific cell types by binding to specific cell-surface receptors.

The linker compounds of the present disclosure may be used to conjugate a cell-targeting or tissue-targeting ligand or other targeting moiety (hereinafter, “targeting agent”) to a payload, which is any substance intended for intracellular or tissue delivery. The targeting agent may be made accessible on the surface of a nanoparticle, exosome, microvesicle, viral vector, other vector, carrier material or other delivery system (“package”) containing a payload for the purpose of delivering the package to a specific target. Alternatively, the targeting agent may be conjugated directly to the payload for direct delivery to the target without the need for formulation into a package. Additionally, the linker compound itself may comprise a targeting agent.

Targeting agents within the scope of the present disclosure include but are not limited to an antibody, antibody fragment, double-chain antibody fragment, or single-chain antibody fragment; other protein, for example, a glycoprotein (e.g., transferrin) and a growth factor; a peptide, cell-penetrating peptide, viral or bacterial epitope, endosomal escape peptide or other endosomal escape agent; a chemical derivative of a peptide, for example 2-[3-(1,3-dicarboxypropyl)-ureido]pentanedioic acid (DUPA); a natural or synthetic carbohydrate, for example, a monosaccharide (e.g., galactose, mannose, N-Acetylgalactosamine [“GalNAc”]), polysaccharide, or a cluster such as lectin binding oligo saccharide, diantennary GalNAc, or triantennary GalNAc; a lipid, for example, a sterol (e.g., cholesterol), phospholipid (e.g., phospholipid ether, phosphatidylcholine, lecithin); a vitamin compound (e.g., tocopherol or folate); immunostimulant (e.g., a CpG oligonucleotide); an amino acid (e.g., arginine-glycine-aspartic acid (“RGD”), a nucleic acid (e.g., an aptamer); an element (e.g., gold); and synthetic molecules (e.g., anisamide and polyethylene glycol). In an embodiment, the targeting agent comprises an aptamer, GalNAc, folate, lipid, cholesterol, or transferrin.

### Drug Delivery Systems

As will be understood by those skilled in the art, regardless of biological target or mechanism of action, therapeutic oligonucleotides must overcome a series of physiological hurdles to access the target cell in an organism (e.g., animal, such as a human, in need of therapy). For example, a therapeutic oligonucleotide generally must avoid clearance in the bloodstream, enter the target cell type, and then enter the cytoplasm, all without eliciting an undesirable immune response. This process is generally considered inefficient, for example, 95% or more of siRNA that enters the endosome in vivo may be degraded in lysosomes or pushed out of the cell without affecting any gene silencing.

To overcome these obstacles, scientists have designed numerous drug delivery vehicles. These vehicles have been used to deliver therapeutic RNAs in addition to small molecule drugs, protein drugs, and other therapeutic molecules. Drug delivery vehicles have been made from materials as diverse as sugars, lipids, lipid-like materials, proteins, polymers, peptides, metals, hydrogels, conjugates, and peptides. Many drug delivery vehicles incorporate aspects from combinations of these groups, for example, some drug delivery vehicles can combine sugars and lipids. In some other examples, drugs can be directly hidden in ‘cell like’ materials that are meant to mimic cells, while in other cases, drugs can be put into, or onto, cells themselves. Drug delivery vehicles can be designed to release drugs in response to stimuli such as pH change, biomolecule concentration, magnetic fields, and heat.

Much work has focused on delivering oligonucleotides such as siRNA to the liver. The dose required for effective siRNA delivery to hepatocytes in vivo has decreased by more than 10,000 fold in the last ten years—whereas delivery vehicles reported in 2006 could require more than 10 mg/kg siRNA to target protein production, with new delivery vehicles target protein production can now be reduced after a systemic injection of 0.001 mg/kg siRNA. The increase in oligonucleotide delivery efficiency can be attributed, at least in part, to developments in delivery vehicles.

Another important advance has been an increased understanding of the way helper components influence delivery. Helper components can include chemical structures added to the primary drug delivery system. Often, helper components can improve particle stability or delivery to a specific organ. For example, nanoparticles can be made of lipids, but the delivery mediated by these lipid nanoparticles can be affected by the presence of hydrophilic polymers and/or hydrophobic molecules. One important hydrophilic polymer that influences nanoparticle delivery is poly(ethylene glycol). Other hydrophilic polymers include non-ionic surfactants. Hydrophobic molecules that affect nanoparticle delivery include cholesterol, 1-2-Distearoyl-sn-glyerco-3-phosphocholine (DSPC), 1-2-di-O-octadecenyl-3-trimethylammonium propane (DOTMA), 1,2-dioleoyl-3-trimethylammonium-propane (DOTAP), and others.

One skilled in the art will appreciate that known delivery vehicles and targeting ligands can generally be adapted for use according to the present disclosure.

Examples of delivery vehicles and targeting ligands, as well as their use, can be found in: Sahay, G., et al. Efficiency of siRNA delivery by lipid nanoparticles is limited by endocytic recycling. Nat Biotechnol, 31: 653-658 (2013); Wittrup, A., et al. Visualizing lipid-formulated siRNA release from endosomes and target gene knockdown. Nat Biotechnol (2015); Whitehead, K. A., Langer, R. & Anderson, D. G. Knocking down barriers: advances in siRNA delivery. Nature reviews. Drug Discovery, 8: 129-138 (2009); Kanasty, R., Dorkin, J. R., Vegas, A. & Anderson, D. Delivery materials for siRNA therapeutics. Nature Materials, 12: 967-977 (2013); Tibbitt, M. W., Dahlman, J. E. & Langer, R. Emerging Frontiers in Drug Delivery. J Am Chem Soc, 138: 704-717 (2016); Akinc, A., et al. Targeted delivery of RNAi therapeutics with endogenous and exogenous ligand-based mechanisms. Molecular therapy: the journal of the American Society of Gene Therapy 18, 1357-1364 (2010); Nair, J. K., et al. Multivalent N-acetylgalactosamine-conjugated siRNA localizes in hepatocytes and elicits robust RNAi-mediated gene silencing. J Am Chem Soc, 136: 16958-16961 (2014); Ostergaard, M. E., et al. Efficient Synthesis and Biological Evaluation of 5′-GalNAc Conjugated Antisense Oligonucleotides. Bioconjugate chemistry (2015); Sehgal, A., et al. An RNAi therapeutic targeting antithrombin to rebalance the coagulation system and promote hemostasis in hemophilia. Nature Medicine, 21: 492-497 (2015); Semple, S. C., et al. Rational design of cationic lipids for siRNA delivery. Nat Biotechnol, 28: 172-176 (2010); Maier, M. A., et al. Biodegradable lipids enabling rapidly eliminated lipid nanoparticles for systemic delivery of RNAi therapeutics. Molecular therapy: the journal of the American Society of Gene Therapy, 21: 1570-1578 (2013); Love, K. T., et al. Lipid-like materials for low-dose, in vivo gene silencing. Proc Nat Acad USA, 107: 1864-1869 (2010); Akinc, A., et al. A combinatorial library of lipid-like materials for delivery of RNAi therapeutics. Nat Biotechnol, 26: 561-569 (2008); Eguchi, A., et al. Efficient siRNA delivery into primary cells by a peptide transduction domain-dsRNA binding domain fusion protein. Nat Biotechnol, 27: 567-571 (2009); Zuckerman, J. E., et al. Correlating animal and human phase Ia/Ib clinical data with CALAA-01, a targeted, polymer-based nanoparticle containing siRNA. Proc Nat Acad USA, 111: 11449-11454 (2014); Zuckerman, J. E. & Davis, M. E. Clinical experiences with systemically administered siRNA-based therapeutics in cancer. Nature Reviews. Drug Discovery, 14: 843-856 (2015); Hao, J., et al. Rapid Synthesis of a Lipocationic Polyester Library via Ring-Opening Polymerization of Functional Valerolactones for Efficacious siRNA Delivery. J Am Chem Soc, 29: 9206-9209 (2015); Siegwart, D. J., et al. Combinatorial synthesis of chemically diverse core-shell nanoparticles for intracellular delivery. Proc Nat Acad USA, 108: 12996-13001 (2011); Dahlman, J. E., et al. In vivo endothelial siRNA delivery using polymeric nanoparticles with low molecular weight. Nat Nano 9, 648-655 (2014); Soppimath, K. S., Aminabhavi, T. M., Kulkarni, A. R. & Rudzinski, W. E. Biodegradable polymeric nanoparticles as drug delivery devices. Journal of controlled release: official journal of the Controlled Release Society 70, 1-20 (2001); Kim, H. J., et al. Precise engineering of siRNA delivery vehicles to tumors using polyion complexes and gold nanoparticles. ACS Nano, 8: 8979-8991 (2014); Krebs, M. D., Jeon, O. & Alsberg, E. Localized and sustained delivery of silencing RNA from macroscopic biopolymer hydrogels. J Am Chem Soc 131, 9204-9206 (2009); Zimmermann, T. S., et al. RNAi-mediated gene silencing in non-human primates. Nature, 441: 111-114 (2006); Dong, Y., et al. Lipopeptide nanoparticles for potent and selective siRNA delivery in rodents and nonhuman primates. Proc Nat Acad USA, 111: 3955-3960 (2014); Zhang, Y., et al. Lipid-modified aminoglycoside derivatives for in vivo siRNA delivery. Advanced Materials, 25: 4641-4645 (2013); Molinaro, R., et al. Biomimetic proteolipid vesicles for targeting inflamed tissues. Nat Mater (2016); Hu, C. M., et al. Nanoparticle biointerfacing by platelet membrane cloaking. Nature, 526: 118-121 (2015); Cheng, R., Meng, F., Deng, C., Klok, H.-A. & Zhong, Z. Dual and multi-stimuli responsive polymeric nanoparticles for programmed site-specific drug delivery. Biomaterials, 34: 3647-3657 (2013); Qiu, Y. & Park, K. Environment-sensitive hydrogels for drug delivery. Advanced Drug Delivery Reviews, 64, Supplement, 49-60 (2012); Mui, B. L., et al. Influence of Polyethylene Glycol Lipid Desorption Rates on Pharmacokinetics and Pharmacodynamics of siRNA Lipid Nanoparticles. Mol Ther Nucleic Acids 2, e139 (2013); Draz, M. S., et al. Nanoparticle-Mediated Systemic Delivery of siRNA for Treatment of Cancers and Viral Infections. Theranostics, 4: 872-892 (2014); Otsuka, H., Nagasaki, Y. & Kataoka, K. PEGylated nanoparticles for biological and pharmaceutical applications. Advanced Drug Delivery Reviews, 55: 403-419 (2003); Kauffman, K. J., et al. Optimization of Lipid Nanoparticle Formulations for mRNA Delivery in vivo with Fractional Factorial and Definitive Screening Designs. Nano Letters, 15: 7300-7306 (2015); Zhang, S., Zhao, B., Jiang, H., Wang, B. & Ma, B. Cationic lipids and polymers mediated vectors for delivery of siRNA. Journal of Controlled Release 123, 1-10 (2007); Illum, L. & Davis, S. S. The organ uptake of intravenously administered colloidal particles can be altered using a non-ionic surfactant (Poloxamer 338). FEBS Letters, 167: 79-82 (1984); Felgner, P. L., et al. Improved Cationic Lipid Formulations for In vivo Gene Therapy. Annals of the New York Academy of Sciences, 772: 126-139 (1995); Meade, B. R. & Dowdy, S. F. Exogenous siRNA delivery using peptide transduction domains/cell penetrating peptides. Advanced Drug Delivery Reviews, 59: 134-140 (2007); Endoh, T. & Ohtsuki, T. Cellular siRNA delivery using cell-penetrating peptides modified for endosomal escape. Advanced Drug Delivery Reviews, 61: 704-709 (2009); and Lee, H., et al. Molecularly self-assembled nucleic acid nanoparticles for targeted in vivo siRNA delivery. Nat Nano, 7: 389-393 (2012).

The following Examples are illustrative and not restrictive. Many variations of the technology will become apparent to those of skill in the art upon review of this disclosure. The scope of the technology should, therefore, be determined not with reference to the Examples, but instead should be determined with reference to the appended claims along with their full scope of equivalents.

### Examples

### Example 1: Gly-Lys bis-(hexanoyl Maleimide) Linker

A Glycine-Lysine dipeptide is prepared by solid phase synthesis and dissolved in aqueous alcohol. 2 equivalents of a solution 6-Maleimidohexanoic acid N-hydroxysuccinimide ester (ECMS) (Creative Biolabs, CAS 55750-63-5) in alcohol are added and the whole stirred for 2 hrs. The resulting N,N,bis-(6-maleimidohexanoyl) glycine-lysine derivative is isolated by preparative chromatography.

### Example 2: Val-Cit bis-(hexanoyl Maleimide) Linker

A Valine-Citrulline dipeptide is prepared by solid phase synthesis and dissolved in aqueous alcohol. 2 equivalents of a solution of 6-Maleimidohexanoic acid N-hydroxysuccinimide ester (ECMS) (Creative Biolabs, CAS 55750-63-5) in alcohol are added and the whole stirred for 2 hrs. The resulting N,N,bis-(6-maleimidohexanoyl) valine-citrulline derivative is isolated by preparative chromatography.

### Example 3: Asp-Lys bis-(hexanoyl Maleimide) Linker

An Aspartate-Lysine iso-dipeptide is prepared by solid phase synthesis and dissolved in aqueous alcohol. 2 equivalents of a solution 6-Maleimidohexanoic acid N-hydroxysuccinimide ester (ECMS) (Creative Biolabs, CAS 55750-63-5) in alcohol are added and the whole stirred for 2 hrs. The resulting N,N,bis-(6-maleimidohexanoyl) aspartate-lysine iso-dipeptide derivative is isolated by preparative chromatography.

### Example 4: Gly-Gly-Val-Lys bis-(hexanoyl Malcimide) Linker

A Glycine-Glycine-Valine-Lysine tetrapeptide is prepared by solid phase synthesis and dissolved in aqueous alcohol. 2 equivalents of a solution 6-Maleimidohexanoic acid N-hydroxysuccinimide ester (ECMS) (Creative Biolabs, CAS 55750-63-5) in alcohol are added and the whole stirred for 2 hrs. The resulting N,N,bis-(6-maleimidohexanoyl) glycine-glycine-valine-lysine derivative is isolated by preparative chromatography.

### Example 5: Gly-Lys bis-(diethylene glycol Maleimide) Linker

A Glycine-Lysine dipeptide is prepared by solid phase synthesis and dissolved in aqueous alcohol. 2 equivalents of a solution of maleimido-di-ethyleneglycol-carboxy-O-pentafluorophenol (Creative Biolabs, MEL-di-EG-OPFP (ADC-L-022)) in alcohol are added and the whole stirred for 2 hrs. The resulting N,N,bis-(carboxydiethylene glycol maleimide) glycine-lysine derivative is isolated by preparative chromatography.

### Example 6: Val-Cit bis-(diethylene glycol Maleimide) Linker

A Valine-Citrulline dipeptide is prepared by solid phase synthesis and dissolved in aqueous alcohol. 2 equivalents of a solution of maleimido-di-ethyleneglycol-carboxy-O-pentafluorophenol (Creative Biolabs, MEL-di-EG-OPFP (ADC-L-022)) in alcohol are added and the whole stirred for 2 hrs. The resulting N,N,bis-(6-maleimidohexanoyl) valine-citrulline derivative is isolated by preparative chromatography.

### Example 7: Asp-Lys bis-(diethylene glycol Maleimide) Linker

An Aspartate-Lysine iso-dipeptide is prepared by solid phase synthesis and dissolved in aqueous alcohol. 2 equivalents of a solution of maleimido-di-ethyleneglycol-carboxy-O-pentafluorophenol (Creative Biolabs, MEL-di-EG-OPFP (ADC-L-022)) in alcohol are added and the whole stirred for 2 hrs. The resulting N,N,bis-(6-maleimidohexanoyl) aspartate-lysine iso-dipeptide derivative is isolated by preparative chromatography.

### Example 8: Gly-Gly-Val-Lys bis-(diethylene glycol Maleimide) Linker

A Glycine-Glycine-Valine-Lysine tetrapeptide is prepared by solid phase synthesis and dissolved in aqueous alcohol. 2 equivalents of a solution of maleimido-di-ethyleneglycol-carboxy-O-pentafluorophenol (Creative Biolabs, MEL-di-EG-OPFP (ADC-L-022)) in alcohol are added and the whole stirred for 2 hrs. The resulting N,N,bis-(6-maleimidohexanoyl) glycine-glycine-valine-lysine derivative is isolated by preparative chromatography.

### Example 9: Gly-Lys bis-(Alkynyl) Linker

A Glycine-Lysine dipeptide is prepared by solid phase synthesis and dissolved in aqueous alcohol. 2 equivalents of a solution of N-hydroxysuccinimidyl hexynoate (Creative BioLabs, 906564-59-8) in alcohol are added and the whole stirred for 2 hrs. The resulting N, N, bis-(5-hexynoyl) glycine-lysine derivative is isolated by preparative chromatography.

### Example 10: Val-Cit bis-(Alkynyl) Linker

A Valine-Citrulline dipeptide is prepared by solid phase synthesis and dissolved in aqueous alcohol. 2 equivalents of a solution of N-hydroxysuccinimidyl hexynoate (Creative BioLabs, 906564-59-8) in alcohol are added and the whole stirred for 2 hrs. The resulting N,N,bis-(5-hexynoyl) valine-citrulline derivative is isolated by preparative chromatography.

### Example 11: Asp-Lys bis-(Alkynyl) Linker

An Aspartate-Lysine iso-dipeptide is prepared by solid phase synthesis and dissolved in aqueous alcohol. 2 equivalents of a solution of N-hydroxysuccinimidyl hexynoate (Creative BioLabs, 906564-59-8) in alcohol are added and the whole stirred for 2 hrs. The resulting N,N,bis-(5-hexynoyl) aspartate-lysine iso-dipeptide derivative is isolated by preparative chromatography.

### Example 12: Gly-Gly-Val-Lys bis-(Alkynyl) Linker

A Glycine-Glycine-Valine-Lysine tetrapeptide is prepared by solid phase synthesis and dissolved in aqueous alcohol. 2 equivalents of a solution solution of N-hydroxysuccinimidyl hexynoate (Creative BioLabs, 906564-59-8) in alcohol are added and the whole stirred for 2 hrs. The resulting N,N,bis-(5-hexynoyl) glycine-glycine-valine-lysine derivative is isolated by preparative chromatography.

### Example 13: Lys-Lys bis-(Alkynyl) Linker With Both Linking Groups on Side Chains

A Lysine-Lysine dipeptide with an N-terminal acetate, and t-boc protected amino groups in the side chains is prepared by solid phase synthesis. The t-boc groups are removed by treatment with methanolic HCl in the presence of anisole. The resulting dipeptide with free e-amino groups is dissolved in aqueous alcohol and treated with 2 equivalents of a solution of N-hydroxysuccinimidyl hexynoate (Creative BioLabs, 906564-59-8) in alcohol and the whole stirred for 2 hrs. The resulting N-acetyl bis-(e-N-5-hexynoyl) lysine-lysine derivative is isolated by preparative chromatography.

### Example 14: Formation of siRNA Dimer Linked by Gly-Gly-Val-Lys bis-(hexanoyl Maleimide)

An siRNA targeting FVII mRNA with a 3′-terminal group is dissolved in aqueous acetonitrile and is treated with 0.5 equivalents of N,N,bis-(6-malcimidohexanoyl) glycine-glycine-valine-lysine and the mixture stirred at room temperature for 3 hrs and then lyophilized. The residue is suspended in aqueous triethyl ammonium bicarbonate buffer, insoluble material is removed by centrifugation, and the desired N,N,bis-(6-maleimidohexanoyl) glycine-glycine-valine-lysine linked dimer of siRNA targeting FVII is isolated by preparative chromatography.

### Example 15: Formation of siRNA-Peptide Hetero-Dimer by Gly-Gly-Val-Lys bis-(hexanoyl Maleimide) Linker

An siRNA targeting FVII mRNA with a 3′-terminal group is dissolved in aqueous acetonitrile and is treated with a solution of 40 equivalents of N,N,bis-(6-maleimidohexanoyl) glycine-glycine-valine-lysine in acetonitrile. The mixture is stirred at room temperature for 3 hrs and then lyophilized. The residue is suspended in aqueous triethyl ammonium bicarbonate buffer, insoluble material is removed by centrifugation, and the N,N,bis-(6-maleimidohexanoyl) glycine-glycine-valine-lysine linker mono-substituted with siRNA targeting FVII is isolated by preparative chromatography.

The transduction domain of HIV-1TAT protein (YGRKKRRQRRR) is prepared by solid phase synthesis with a N-terminal amino function and a C-terminal cysteine residue. After purification the end product is dissolved in aqueous dimethyformamide (DMF) and added to a solution in DMF of the mono-substituted N,N,bis-(6-maleimidohexanoyl) glycine-glycine-valine-lysine linker prepared above.

The mixture is stirred at room temperature overnight and then evaporated to dryness. The desired siRNA: N,N,bis-(6-maleimidohexanoyl) glycine-glycine-valine-lysine: peptide heterodimer is isolated by preparative chromatography.

### Example 16: Formation of a Trivalent Linker

N,N,bis-(6-maleimidohexanoyl) glycine-lysine (MGKM) prepared in Example 1 is dissolved in aqueous acetonitrile and added to a 40-fold deficiency of 1-thioglycerol in the same solvent. After 2 hrs the desired mono-thiolglycerol derivative of MGKM is isolated by chromatography.

1. A homo-bivalent linker compound comprising identical functional groups at either end, wherein said functional groups are joined by a linking group comprising at least one amide bond.

2. The homo-bivalent linker compound of claim 1, wherein the at least one amide bond is a eupeptide bond.

3. The homo-bivalent linker compound of claim 1, wherein the at least one amide bond is an isopeptide bond.

4. The homo-bivalent linker compound of claim 1, wherein the at least one amide bond is formed from the joining of two amino acids.

5. The homo-bivalent linker compound of claim 4, wherein each of the amino acids is independently naturally occurring or non-naturally occurring.

6. The homo-bivalent linker compound of claim 4, wherein each of the amino acids is independently an alpha, beta, gamma, or delta amino acid.

7. The homo-bivalent linker compound of claim 4, wherein at least one of the amino acids is an alpha amino acid; or wherein each of the amino acids is an alpha amino acid.

8. The homo-bivalent linker compound of claim 4, wherein at least one of the amino acids is a proteogenic amino acid; or wherein each of the amino acids is a proteogenic amino acid.

9. The homo-bivalent linker compound of any of claims 1 to 8, wherein the identical functional groups are maleimide, azide, alkyne, activated carboxyl or amine.

10. The homo-bivalent linker compound of any of claims 1 to 9, wherein the compound comprises Structure 1:
(X)-<--->-□-<--->-(X) (Structure 1);
wherein,
(X) is a function group;
each <---> is independently a spacer group, which may be present or absent; and
□ is a linking group comprising at least one amide bond.

11. The homo-bivalent linker compound of claim 10, wherein X is a maleimide, azide, alkyne, activated carboxyl or amine.

12. The homo-bivalent linker compound of claim 10 or 11, wherein each spacer group <---> that is present in the compound is, independently, alkyl, alkoxy, cyclyl, heterocyclyl, aryl, heteroaryl, or substituted versions thereof.

13. The homo-bivalent linker compound of claim 12, wherein each spacer group <---> that is present in the compound is, independently, C₁₋₁₀ alkyl, C₁₋₁₀ alkoxy, 5-10 membered aryl, 5-10 membered heteroaryl, 5-10 membered heterocyclyl, (C₁₋₁₀ alkyl)-(5-10 membered aryl), (C₁₋₁₀ alkyl)-(5-10 membered heteroaryl), or (C₁₋₁₀ alkyl)-(5-10 membered heterocyclyl).

14. The homo-bivalent linker compound of claim 13, wherein each spacer group <---> that is present in the compound is, independently, C₂ to C₆ alkyl, ethylene glycol, triethylene glycol, or 1,4-phenylene.

15. The homo-bivalent linker compound of any of claims 10 to 14, wherein the linking group □ comprises 1, 2, 3, or more than 3 amide bonds.

16. The homo-bivalent linker compound of claim 15, wherein the linking group □ comprises 1, 2, or 3 amide bonds; optionally wherein the linking group □ comprises at least one amide bond formed from the linkage of two amino acids.

17. The homo-bivalent linker compound of claim 16, wherein the linking group □ comprises:
(i) one amide bond formed from the linkage of two amino acids;
(ii) two amide bonds formed from the linkage of three amino acids; or
(iii) three amide bonds formed from the linkage of four amino acids.

18. The homo-bivalent linker compound of any of claims 10 to 17, wherein each of the amide bonds is, independently, a eupeptide bond or an isopeptide bond.

19. The homo-bivalent linker compound of any of claims 16 to 18, wherein at least one amino acid is Glycine.

20. The homo-bivalent linker compound of any of claims 16 to 19, wherein at least one amino acid is Alanine.

21. The homo-bivalent linker compound of any of claims 16 to 20, wherein at least one amino acid is Proline.

22. The homo-bivalent linker compound of any of claims 16 to 21, wherein at least one amino acid is Valine.

23. The homo-bivalent linker compound of any of claims 16 to 22, wherein at least one amino acid is Lysine.

24. The homo-bivalent linker compound of any of claims 16 to 23, wherein at least one amino acid is Aspartic Acid.

25. The homo-bivalent linker compound of any of claims 16 to 24, wherein at least one amino acid is Citrulline.

26. The homo-bivalent linker compound of any of claims 16 to 25, wherein at least one amino acid is Beta-alanine.

27. The homo-bivalent linker compound of any of claims 10 to 26, wherein the linking group □ comprises Structure 2:
R-Aₐ-B_{b}-C_{c}-Dₐ-R′ Structure 2
wherein:
R is H, or is absent;
R′is OH, or is absent;
each of a, b, c, and d is independently 0 or 1, with the proviso that the sum of a+b+c+d is greater than or equal to 2; and
each of A, B, C and D independently comprises Structure 3:
[N▴ [symbol] -(CH [symbol] )_{w}-(CH [symbol] )ₓ-(CH [symbol] )_{y}-(CH [symbol] )_{z}-CO-▾] (Structure 3)
wherein:
each of w, x, y, and z are independently 0 or 1, with the proviso that the sum of w+x+y+z is greater than or equal to 1;
each ▴ is independently H, H₂, alkyl, alkoxy, alkyl carboxy, alkyl carboxamide, alkyl amino, alkyl sulfate, aryl, aryl carboxy, aryl carboxamide, aryl amino, aryl sulfate, or is absent;
each of [symbol] , [symbol] , [symbol] , [symbol] , and [symbol] is independently present or absent, and if present designates a terminus of a cyclic group as follows: [symbol]
designates the N in N▴ [symbol] as a terminus; [symbol]
designates the C in (CH [symbol] )_{w} as a terminus; [symbol]
designates the C in (CH [symbol] )ₓ as a terminus; [symbol]
designates the C in (CH [symbol] )_{y} as a terminus; and [symbol]
designates the C in (CH [symbol] )_{z} as a terminus;
with the proviso that each Structure 3 independently contains zero, one or two cyclic groups, the termini of each cyclic group being selected from: [symbol]
as a first terminus and [symbol] , [symbol] , [symbol] , or [symbol] as a second terminus; [symbol]
as a first terminus and [symbol] , [symbol] , or [symbol] as a second terminus; [symbol]
as a first terminus and [symbol] or [symbol] as a second terminus; or [symbol]
as a first terminus and [symbol] as a second terminus;
with the further proviso that:
if [symbol] is present, then [symbol] is absent;
if [symbol] is present, then [symbol] is absent;
if [symbol] is present, then [symbol] is absent;
if [symbol] is present, then [symbol] is absent;
each cyclic group that is present in Structure 3 further comprises, in addition to its respective termini, a middle section between the termini, Y; and each Y is independently alkyl, alkoxy, alkyl carboxy, alkyl carboxamide, alkyl amino, or alkyl sulfate;
each of [symbol] , [symbol] , [symbol] , and [symbol] are independently present or absent, and if present are H, OH, alkyl, alkyl carboxy, alkyl carboxamide, alkyl amino, alkoxy, thioalkyl, alkylthioalkyl, aryl, or heteroaryl;
each of [symbol] , [symbol] , [symbol] , and [symbol] are, where present, optionally bonded to a functional end group X, with or without an intervening spacer group ---;
each ▾ is independently OH, alkyl, alkoxy, alkyl carboxy, alkyl carboxamide, alkyl amino, alkyl sulfate, aryl, aryl carboxy, aryl carboxamide, aryl amino, aryl sulfate, or is absent;
with the proviso that homo-bivalent linker compound contains a total of only two functional end groups X, in keeping with the compound being a homo-bivalent linker compound; and
Structure 2 optionally comprises at least one amide bond that is optionally formed from the linkage of two amino acids.

28. The homo-bivalent linker compound of claim 27, wherein X is maleimide, azide, alkyne, activated carboxyl or amine.

29. The homo-bivalent linker compound of claim 27 or 28, wherein each spacer group --- is, independently, alkyl, alkoxy, cyclyl, heterocyclyl, aryl, heteroaryl, or substituted versions thereof.

30. The homo-bivalent linker compound of claim 29, wherein each spacer group --- is, independently, C₁₋₁₀ alkyl, C₁₋₁₀ alkoxy, 5-10 membered aryl, 5-10 membered heteroaryl, 5-10 membered heterocyclyl, (C₁₋₁₀ alkyl)-(5-10 membered aryl), (C₁₋₁₀ alkyl)-(5-10 membered heteroaryl), or (C₁₋₁₀ alkyl)-(5-10 membered heterocyclyl).

31. The homo-bivalent linker compound of claim 30, wherein each spacer group --- is, independently, C₂ to C₆ alkyl, ethylene glycol, triethylene glycol, or 1,4-phenylene.

32. The homo-bivalent linker compound of any of claims 27 to 31, wherein at least one amide bond is a eupeptide bond.

33. The homo-bivalent linker compound of any of claims 27 to 31, wherein at least one amide bond is an isopeptide bond.

34. The homo-bivalent linker compound of any of claims 27 to 33, wherein at least one amide bond is formed from the joining of two amino acids.

35. The homo-bivalent linker compound of claim 34, wherein each of the amino acids is independently naturally occurring or non-naturally occurring.

36. The homo-bivalent linker compound of claim 34, wherein each of the amino acids is independently an alpha, beta, gamma, or delta amino acid.

37. The homo-bivalent linker compound of claim 34, wherein at least one of the amino acids is an alpha amino acid; or wherein each of the amino acids is an alpha amino acid.

38. The homo-bivalent linker compound of claim 34, wherein at least one of the amino acids is a proteogenic amino acid; or wherein each of the amino acids is a proteogenic amino acid.

39. The homo-bivalent linker compound of claim 27, wherein the compound comprises a eupeptide bond formed by the joining of Glycine to Glycine, wherein;
with respect to Structure 2:
each of the spacer groups <---> are present;
R and R′are absent;
a and b are 1;
c and d are 0;
with respect to element A, Structure 3:
w is 1;
x, y, and z are 0;
▴ is H; [symbol]
is H; [symbol]
and [symbol] are absent; and
▾ is absent;
with respect to element B, Structure 3:
w is 1;
x, y, and z are 0;
▴ is H; [symbol]
is H; [symbol]
and [symbol] are absent; and
▾ is absent;
with the result that the homo-bivalent linker compound comprises Structure 4:
X----NH—(CH₂)—CO—NH—(CH₂)—CO----X (Structure 4).

40. The homo-bivalent linker compound of claim 27, wherein the compound comprises a eupeptide bond formed by the joining of Glycine to Alanine, wherein:
with respect to Structure 2:
each of the spacer groups <---> are present R and R′ are absent;
a and b are 1;
c and d are 0;
with respect to element A, Structure 3;
w is 1;
x, y, and z are 0;
▴ is H; [symbol]
is H; [symbol]
and [symbol] are absent; and
▾ is absent;
with respect to element B, Structure 3:
w is 1;
x, y, and z are 0;
▴ is H; [symbol]
is methyl; [symbol]
and [symbol] are absent; and
▾ is absent;
with the result that the homo-bivalent linker compound comprises Structure 5:
X----NH—(CH₂)—CO—NH—(CHMethyl)-CO----X (Structure 5).

41. The homo-bivalent linker compound of claim 27, wherein the compound comprises a eupeptide bond formed by the joining of Glycine to Proline, wherein:
with respect to Structure 2:
each of the spacer groups <---> are present R and R′ are absent;
a and b are 1;
c and d are 0;
with respect to element A, Structure 3:
w is 1;
x, y, and z are 0;
▴ is H; [symbol]
is H; [symbol]
and [symbol] are absent; and
▾ is absent;
with respect to element B, Structure 3:
w is 1;
x, y, and z are 0;
▴ is absent; [symbol]
and [symbol] are present;
Y is propyl and the cyclic group is pyrrolidine; and [symbol]
, [symbol] , and ▾ are absent;
with the result that the homo-bivalent linker compound comprises Structure 6:

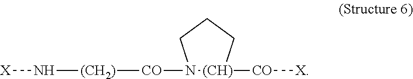

42. The homo-bivalent linker compound of claim 27, wherein the compound comprises a eupeptide bond formed by the joining of Glycine to Valine, wherein:
with respect to Structure 2:
each of the spacer groups <---> are present R and R′ are absent;
a and b are 1;
c and d are 0;
with respect to element A, Structure 3:
w is 1;
x, y, and z are 0;
▴ is H; [symbol]
is H; [symbol]
and [symbol] are absent; and
▾ is absent;
with respect to element B, Structure 3:
w is 1;
x, y, and z are 0;
▴ is H; [symbol]
is isopropyl; [symbol]
and [symbol] are absent; and
▾ is absent;
with the result that the homo-bivalent linker compound comprises Structure 7:
X----NH—(CH₂)—CO—NH—(CH-isopropyl)-CO----X (Structure 7).

43. The homo-bivalent linker compound of claim 27, wherein the compound comprises a eupeptide bond formed by the joining of Glycine to Lysine, wherein:
with respect to Structure 2:
each of the spacer groups <---> are present R and R′ are absent;
a and b are 1;
c and d are 0;
with respect to element A, Structure 3:
w is 1;
x, y, and z are 0;
▴ is H; [symbol]
is H; [symbol]
and [symbol] are absent; and
▾ is absent;
with respect to element B, Structure 3:
w is 1;
x, y, and z are 0;
▴ is H; [symbol]
is butylamino; [symbol]
and [symbol] are absent; and
▾ is absent;
with the result that the homo-bivalent linker compound comprises Structure 8:

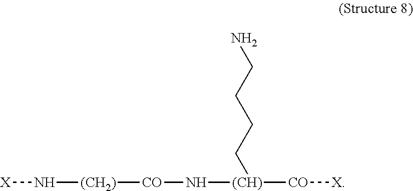

44. The homo-bivalent linker compound of claim 27, wherein the compound comprises a eupeptide bond formed by the joining of Glycine to Lysine, wherein:
with respect to Structure 2:
each of the spacer groups <---> are present R and R′ are absent;
a and b are 1;
c and d are 0;
with respect to element A, Structure 3:
w is 1;
x, y, and z are 0;
▴ is H; [symbol]
is H; [symbol]
and [symbol] are absent; and
▾ is absent;
with respect to element B, Structure 3:
w is 1;
x, y, and z are 0;
▴ is H; [symbol]
is butylamino bonded to an X; [symbol]
and [symbol] are absent; and
▾ is OH,
with the result that the bivalent linker compound comprises Structure 9:

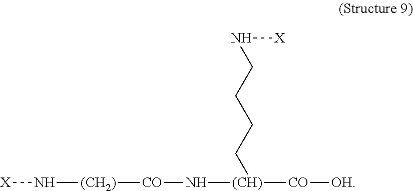

45. The homo-bivalent linker compound of any of claim 27, wherein the compound comprises a eupeptide bond formed by the joining of Glycine to Aspartic Acid, wherein:
with respect to Structure 2:
each of the spacer groups <---> are present R and R′ are absent;
a and b are 1;
c and d are 0;
with respect to element A, Structure 3:
w is 1;
x, y, and z are 0;
▴ is H; [symbol]
is H; [symbol]
and [symbol] are absent; and
▾ is absent;
with respect to element B, Structure 3:
w is 1;
x, y, and z are 0;
▴ is H; [symbol]
is acetate; [symbol]
and [symbol] are absent; and
▾ is absent;
with the result that the homo-bivalent linker compound comprises Structure 10:

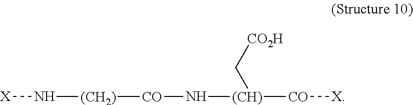

46. The homo-bivalent linker compound of claim 27, wherein the compound comprises a eupeptide bond and an isopeptide bond formed by the joining of Glycine, Aspartic Acid, and Lysine, wherein:
with respect to Structure 2:
each of the spacer groups <---> are present R and R′ are absent;
a, b, and c are 1;
d is 0;
with respect to element A, Structure 3:
w is 1;
x, y, and z are 0; [symbol]
is H; [symbol]
and [symbol] are absent; and
▾ is absent;
with respect to element B, Structure 3:
w is 1;
x, y, and z are 0;
▴ is H; [symbol]
is acetyl; [symbol]
and [symbol] are absent; and
▾ is OH;
with respect to element C, Structure 3:
w is 1;
x, y, and z are 0;
▴ is H; [symbol]
is butylamino bonded to an X; [symbol]
and [symbol] are absent; and
▾ is OH;
with the result that the homo-bivalent linker compound comprises Structure 11:

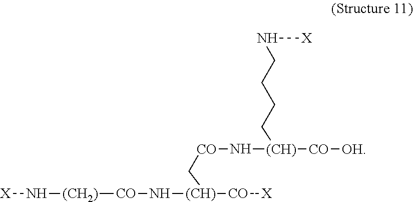

47. The homo-bivalent linker compound of claim 27, wherein the compound comprises a eupeptide bond formed by the joining of Glycine to Beta-Alanine, wherein:
with respect to Structure 2:
each of the spacer groups <---> are present R and R′ are absent;
a and b are 1;
c and d are 0;
with respect to element A, Structure 3:
w is 1;
x, y, and z are 0;
▴ is H; [symbol]
is H; [symbol]
and [symbol] are absent; and
▾ is absent;
with respect to element B, Structure 3:
w is 1;
x is 1;
y and z are 0;
▴ is H; [symbol]
is H; [symbol]
is H; [symbol]
, [symbol] , and [symbol] are absent; and
▾ is absent;
with the result that the homo-bivalent linker compound comprises Structure 12:
X----NH—(CH₂)—CO—NH—(CH₂)—(CH₂)—CO----X (Structure 12).

48. The homo-bivalent linker compound of claim 27, wherein the compound comprises a eupeptide bond formed by the joining of Valine to Citrulline, wherein:
with respect to Structure 2:
each of the spacer groups <---> are present R and R′ are absent;
a and b are 1;
c and d are 0;
with respect to element A, Structure 3:
w is 1;
x, y, and z are 0;
▴ is H; [symbol]
is isopropyl; [symbol]
and [symbol] are absent; and
▾ is absent;
with respect to element B, Structure 3:
w is 1;
x, y, and z are 0;
▴ is H; [symbol]
is propylcarbamoylamino; [symbol]
and [symbol] are absent; and
▾ is absent;
with the result that the homo-bivalent linker compound comprises Structure 13:

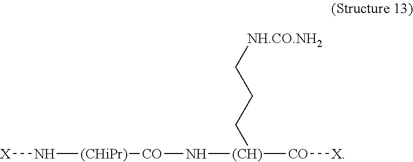

49. The homo-bivalent linker compound of claim 27, wherein the compound comprises a eupeptide bond formed by joining Lysine to Lysine, wherein:
with respect to Structure 2:
each of the spacer groups <---> are present R is H;
R′ is OH;
a and b are 1;
c and d are 0;
with respect to element A, Structure 3:
w is 1;
x, y, and z are 0;
▴ is H; [symbol]
is butylamino bonded to an X; [symbol]
and [symbol] are absent; and
▾ is absent;
with respect to element B, Structure 3:
w is 1;
x, y, and z are 0;
▴ is H; [symbol]
is butylamino bonded to an X; [symbol]
and [symbol] are absent; and
▾ is absent;
with the result that the bivalent linker compound comprises Structure 14:

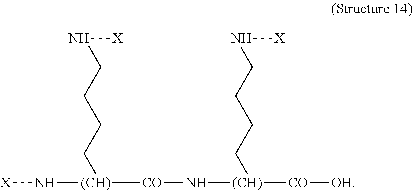

50. A branched linker compound of Structure 15: 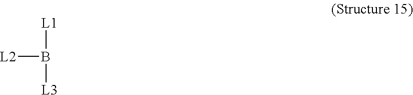
wherein:
B is a trivalent moiety;
each of L1, L2 and L3 is a branch group; and
at least one of L1, L2 and L3 is formed by the joining of B to a homo-bivalent linker compound of any of claims 1 to 49; optionally at least two of L1, L2 and L3 are, independently, formed by the joining of B to a homo-bivalent linker compound of any of claims 1 to 49; optionally each of L1, L2 and L3 are, independently, formed by the joining of B to a homo-bivalent linker compound of any of claims 1 to 49.

51. The linker compound of any of claims 1-50, wherein the compound is at least 75, 80, 85, 90, 95, 96, 97, 98, 99, or 100% pure.

52. The linker compound of any of claims 1-50, wherein the compound is about 85-95% pure.

53. The linker compound of any of claims 1-50, wherein the compound is greater than or equal to 75% pure; greater than or equal to 85% pure; or greater than or equal to 95% pure.

54. A multi-conjugate comprising two or more biological moieties joined together by covalent bonds, wherein at least one covalent bond within the multi-conjugate is formed by reaction with a linker compound of any of claims 1 to 50.

55. The multi-conjugate of claim 54, wherein each of the biological moieties is joined to another biological moiety by a linker compound of any of claims 1 to 50.

56. The multi-conjugate of claim 54 or 55, wherein the multi-conjugate comprises two, three, four, five, or six biological moieties.

57. The multi-conjugate of any of claims 54 to 56, wherein each biological moiety is, independently, a nucleic acid, peptide, protein, lipid, carbohydrate, carboxylic acid, vitamin, steroid, lignin, small molecule, organometallic compound, or a derivative of any of the foregoing.

58. The multi-conjugate of any of claims 54 to 57, wherein at least two biological moieties are oligonucleotides; optionally the at least two oligonucleotides are adjacent one another in the multi-conjugate; and optionally each of the oligonucleotides is 15-30, 17-27, 19-26, or 20-25 nucleotides in length.

59. The multi-conjugate of any of claims 54 to 58, wherein at least one of the biological moieties is a double-stranded RNA; optionally an siRNA, a saRNA, or a miRNA.

60. The multi-conjugate of any of claims 54 to 59, wherein at least one of the biological moieties is a single-stranded RNA, optionally an antisense oligonucleotide.

61. The multi-conjugate of claim 59, wherein each of the biological moieties is a double-stranded siRNA.

62. The multi-conjugate of any of claims 54 to 60, wherein at least one biological moiety is a protein, a peptide, or a derivative thereof.

63. The multi-conjugate of any of claims 54 to 62, having one or more covalent bonds formed by reaction with a homo-bivalent linker compound having maleimide functional groups, each of which, upon reaction, is independently

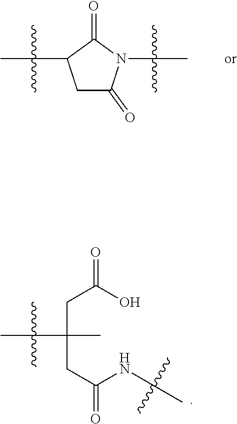 [SMILES: *C1CC(=O)N(*)C1=O ; *NC(=O)CC(*)(C)CC(=O)O]

64. A method for synthesizing a multi-conjugate according to any of claims 54 to 63, comprising the steps of reacting a homo-bivalent linker compound according to any of claims 1 to 50 with a first and a second biological moiety, under reaction conditions that promote the formation of a covalent bond between the first biological moiety and the linker compound and a covalent bond between the second biological moiety and the linker compound.

65. The method of claim 64, wherein the first biological moiety and the second biological moiety are the same and the coupling of each of the biological moieties to the homo-bivalent linker compound is performed simultaneously.

66. The method of claim 64, wherein the first biological moiety and the second biological moiety are different and the coupling of each of the biological moieties to the homo-bivalent linker compound is performed sequentially under reaction conditions that substantially favor the formation of an isolatable intermediate comprising the homo-bivalent linker monosubstituted with the first biological moiety and substantially prevent dimerization of the first biological moiety.

67. The method of claim 66, wherein the coupling of the homo-bivalent linker compound to the first biological moiety is carried out in a dilute solution of the first biological moiety with a stoichiometric excess of the homo-bivalent linker compound.

68. The method of claim 67, wherein the coupling of the homo-bivalent linker compound to the first biological moiety is carried out with a molar excess of the homo-bivalent linker compound of at least about 5, 10, 15, 20, 25, 30, 35, 40, 45, 50, or 100.

69. The method of claim 67, wherein the coupling of the homo-bivalent linker compound to the first biological moiety is carried out with a molar excess of the homo-bivalent linker compound of about 5, 10, 15, 20, 25, 30, 35, 40, 45, 50, or 100.

70. The method of any of claims 66 to 69, wherein the coupling of the homo-bivalent linker compound to the first biological moiety is carried out in a solution comprising water and a water miscible organic co-solvent.

71. The method of claim 70, wherein the water miscible organic co-solvent comprises DMF, NMP, DMSO, alcohol, or acetonitrile.

72. The method of claim 70 or 71, wherein the water miscible organic co-solvent comprises about 10, 15, 20, 25, 30, 40, or 50% (v/v) of the solution.

73. The method of any of claims 70 to 72, wherein the coupling of the homo-bivalent linker compound to the first biological moiety is carried out at a pH of below about 7, 6, 5, or 4.

74. The method of any of claims 70 to 72, wherein the coupling of the homo-bivalent linker compound to the first biological moiety is carried out at a pH of about 7, 6, 5, or 4.

75. The method of any of claims 66 to 69, wherein the coupling of the homo-bivalent linker compound to the first biological moiety is carried out in a solution comprising an anhydrous organic solvent.

76. The method of claim 75, wherein the anhydrous organic solvent comprises dichloromethane, DMF, DMSO, THF, dioxane, pyridine, alcohol, or acetonitrile.

77. The method of any of claims 64 to 76, wherein the yield of the multi-conjugate is at least 75, 80, 85, 90, 95, 96, 97, 98, 99, or 100%.

78. The method of any of claims 64 to 77, wherein the purity of the multi-conjugate is at least 75, at least 75, 80, 85, 90, 95, 96, 97, 98, 99, or 100%.

79. A compound comprising a homo-bivalent linker of any of claims 1 to 49 substituted on one end by a biological moiety, wherein the other end of the homo-bivalent linker is unsubstituted, and wherein the compound is at least 75%, 80, 85, 90, 95, 96, 97, 98, 99, or 100% pure.

80. The compound of claim 79, wherein the biological moiety is a nucleic acid, peptide, protein, lipid, carbohydrate, carboxylic acid, vitamin, steroid, lignin, small molecule, organometallic compound, or a derivative of any of the foregoing.

81. A pharmaceutical composition comprising the multi-conjugate of any of claims 54 to 63.

82. A composition comprising the multi-conjugate of any of claims 54 to 63 for use in the manufacture of a medicament.

83. A method for treating a subject in need of treatment to ameliorate, cure, or prevent the onset of a disease or disorder, the method comprising administering to the subject an effective amount of the multi-conjugate of any of claims 54 to 63.

84. A method for modulating gene expression in a cell, in vitro or in vivo, the method comprising delivering to the cell an effective amount of a multi-conjugate according to any of claims 54 to 63, wherein the multi-conjugate comprises at least one biological moiety that has the effect of modulating gene expression.

85. The method of claim 84, wherein the at least one biological moiety silences or reduces gene expression; optionally, wherein the at least one biological moiety is siRNA, miRNA, or an antisense oligonucleotide.

86. The method of claim 84, wherein the at least one biological moiety activates or increases gene expression; optionally wherein the at least one biological moiety is saRNA.

87. A method for delivering, in vitro or in vivo, two or more biological moieties to a cell per internalization event, comprising administering to the cell a multi-conjugate according to any of claims 54 to 63.

88. The method of claim 87, wherein the multi-conjugate is formulated in a lipid nanoparticle.

89. The method of claim 87, wherein the multi-conjugate is packaged in a viral vector.

90. The method of claim 87, wherein the multi-conjugate comprises a cell- or tissue-targeting ligand.

91. The method of any of claims 83-90, wherein the multi-conjugate comprises 3 or more biological moieties in a predetermined stoichiometric ratio.

92. A method of treating a disease or condition in a subject comprising the step of administering to the subject an effective amount of a pharmaceutical composition comprising an active pharmaceutical ingredient joined by a covalent bond formed by reaction with a linker compound of any of claims 1 to 50.

93. A homo-bivalent linker compound comprising:

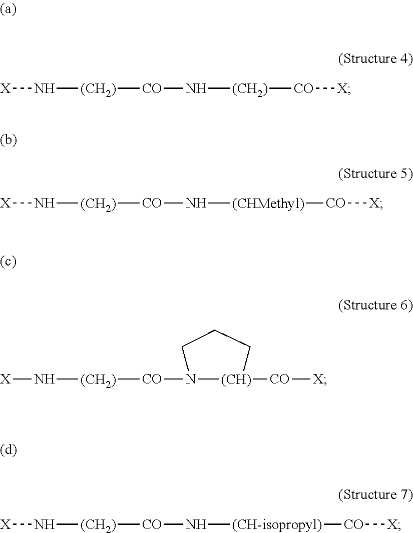

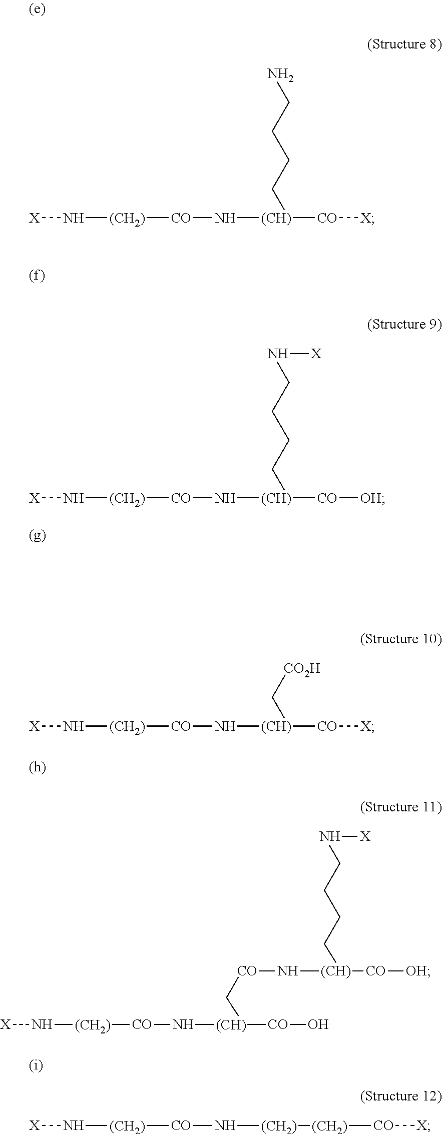

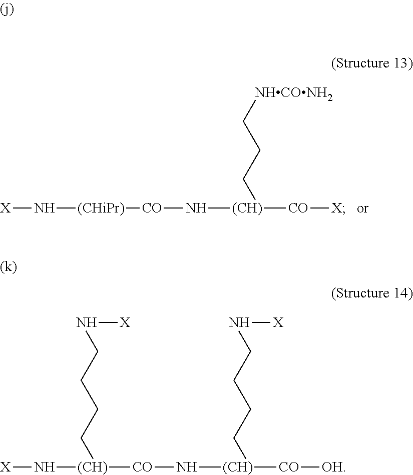

94. The method of claim 64, wherein the first and second biological moiety are each independently a nucleic acid, peptide, protein, lipid, carbohydrate, carboxylic acid, vitamin, steroid, lignin, small molecule, organometallic compound, or a derivative of any of the foregoing.
